SEQUENCE LISTING (4 sequences)

SEQ ID NO: 1 - DNA, 1753 bp, Artificial Sequence
  keratin 7 cDNA sequence
cagccccgcccctacctgtggaagcccagccgcccgctcccgcggataaaaggcgcggagtgtccccgaggtcagcgagtgcgcgctcctcctcgcccgccgctaggtccatcccggcccagccaccatgtccatccacttcagctccccggtattcacctcgcgctcagccgccttctcgggccgcggcgcccaggtgcgcctgagctccgctcgccccggcggccttggcagcagcagcctctacggcctcggcgcctcacggccgcgcgtggccgtgcgctctgcctatgggggcccggtgggcgccggcatccgcgaggtcaccattaaccagagcctgctggccccgctgcggctggacgccgacccctccctccagcgggtgcgccaggaggagagcgagcagatcaagaccctcaacaacaagtttgcctccttcatcgacaaggtgcggtttctggagcagcagaacaagctgctggagaccaagtggacgctgctgcaggagcagaagtcggccaagagcagccgcctcccagacatctttgaggcccagattgctggccttcggggtcagcttgaggcactgcaggtggatgggggccgcctggaggcggagctgcggagcatgcaggatgtggtggaggacttcaagaataagtacgaagatgaaattaaccaccgcacagctgctgagaatgagtttgtggtgctgaagaaggatgtggatgctgcctacatgagcaaggtggagctggaggccaaggtggatgccctgaatgatgagatcaacttcctcaggaccctcaatgagacggagttgacagagctgcagtcccagatctccgacacatctgtggtgctgtccatggacaacagtcgctccctggacctggacggcatcatcgctgaggtcaaggcgcagtatgaggagatggccaaatgcagccgggctgaggctgaagcctggtaccagaccaagtttgagaccctccaggcccaggctgggaagcatggggacgacctccggaatacccggaatgagatttcagagatgaaccgggccatccagaggctgcaggctgagatcgacaacatcaagaaccagcgtgccaagttggaggccgccattgccgaggctgaggagcgtggggagctggcgctcaaggatgctcgtgccaagcaggaggagctggaagccgccctgcagcggggcaagcaggatatggcacggcagctgcgtgagtaccaggaactcatgagcgtgaagctggccctggacatcgagatcgccacctaccgcaagctgctggagggcgaggagagccggttggctggagatggagtgggagccgtgaatatctctgtgatgaattccactggtggcagtagcagtggcggtggcattgggctgaccctcgggggaaccatgggcagcaatgccctgagcttctccagcagtgcgggtcctgggctcctgaaggcttattccatccggaccgcatccgccagtcgcaggagtgcccgcgactgagccgcctcccaccactccactcctccagccaccacccacaatcacaagaagattcccacccctgcctcccatgcctggtcccaagacagtgagacagtctggaaagtgatgtcagaatagcttccaataaagcagcctcattctgaggcctgagtgatccacgtgaaaaaaaaaaaaaaaaaaaaaaaaaaaaaaaaaaaaaaaaaaaaaaaaaaa

SEQ ID NO: 2 - DNA, 1817 bp, Artificial Sequence
  keratin 20 cDNA sequence
caaccatcctgaagctacaggtgctccctcctggaatctccaatggatttcagtcgcagaagcttccacagaagcctgagctcctccttgcaggcccctgtagtcagtacagtgggcatgcagcgcctcgggacgacacccagcgtttatgggggtgctggaggccggggcatccgcatctccaactccagacacacggtgaactatgggagcgatctcacaggcggcggggacctgtttgttggcaatgagaaaatggccatgcagaacctaaatgaccgtctagcgagctacctagaaaaggtgcggaccctggagcagtccaactccaaacttgaagtgcaaatcaagcagtggtacgaaaccaacgccccgagggctggtcgcgactacagtgcatattacagacaaattgaagagctgcgaagtcagattaaggatgctcaactgcaaaatgctcggtgtgtcctgcaaattgataatgctaaactggctgctgaggacttcagactgaagtatgagactgagagaggaatacgtctaacagtggaagctgatctccaaggcctgaataaggtctttgatgacctaaccctacataaaacagatttggagattcaaattgaagaactgaataaagacctagctctcctcaaaaaggagcatcaggaggaagtcgatggcctacacaagcatctgggcaacactgtcaatgtggaggttgatgctgctccaggcctgaaccttggcgtcatcatgaatgaaatgaggcagaagtatgaagtcatggcccagaagaaccttcaagaggccaaagaacagtttgagagacagactgcagttctgcagcaacaggtcacagtgaatactgaagaattaaaaggaactgaggttcaactaacggagctgagacgcacctcccagagccttgagatagaactccagtcccatctcagcatgaaagagtctttggagcacactctagaggagaccaaggcccgttacagcagccagttagccaacctccagtcgctgttgagctctctggaggcccaactgatgcagattcggagtaacatggaacgccagaacaacgaataccatatccttcttgacataaagactcgacttgaacaggaaattgctacttaccgccgccttctggaaggagaagacgtaaaaactacagaatatcagttaagcaccctggaagagagagatataaagaaaaccaggaagattaagacagtcgtgcaagaagtagtggatggcaaggtcgtgtcatctgaagtcaaagaggtggaagaaaatatctaaatagctaccagaaggagatgctgctgaggttttgaaagaaatttggctataatcttatctttgctccctgcaagaaatcagccataagaaagcactattaatactctgcagtgattagaaggggtggggtggcgggaatcctatttatcagactctgtaattgaatataaatgttttactcagaggagctgcaaattgcctgcaaaaatgaaatccagtgagcactagaatatttaaaacatcattactgccatctttatcatgaagcacatcaattacaagctgtagaccacctaatatcaatttgtaggtaatgttcctgaaaattgcaatacatttcaattatactaaacctcacaaagtagaggaatccatgtaaattgcaaataaaccactttctaattttttcctgtttctgaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 2090 bp, Artificial Sequence
  caudal type homeobox 2 (CDX2) cDNA sequence
gggaccgcctcggaggcagaagagccgcgaggagccagcggagcaccgcgggctggggcgcagccacccgccgctcctcgagtcccctcgcccctttcccttcgtgccccccggcagcctccagcgtcggtccccaggcagcatggtgaggtctgctcccggaccctcgccaccatgtacgtgagctacctcctggacaaggacgtgagcatgtaccctagctccgtgcgccactctggcggcctcaacctggcgccgcagaacttcgtcagccccccgcagtacccggactacggcggttaccacgtggcggccgcagctgcagcggcagcgaacttggacagcgcgcagtccccggggccatcctggccggcagcgtatggcgccccactccgggaggactggaatggctacgcgcccggaggcgccgcggccgccgccaacgccgtggctcacggcctcaacggtggctccccggccgcagccatgggctacagcagccccgcagactaccatccgcaccaccacccgcatcaccacccgcaccacccggccgccgcgccttcctgcgcttctgggctgctgcaaacgctcaaccccggccctcctgggcccgccgccaccgctgccgccgagcagctgtctcccggcggccagcggcggaacctgtgcgagtggatgcggaagccggcgcagcagtccctcggcagccaagtgaaaaccaggacgaaagacaaatatcgagtggtgtacacggaccaccagcggctggagctggagaaggagtttcactacagtcgctacatcaccatccggaggaaagccgagctagccgccacgctggggctctctgagaggcaggttaaaatctggtttcagaaccgcagagcaaaggagaggaaaatcaacaagaagaagttgcagcagcaacagcagcagcagccaccacagccgcctccgccgccaccacagcctccccagcctcagccaggtcctctgagaagtgtcccagagcccttgagtccggtgtcttccctgcaagcctcagtgcctggctctgtccctggggttctggggccaactgggggggtgctaaaccccaccgtcacccagtgacccaccgggttctgcagcggcagagcaattccaggctgagccatgaggagcgtggactctgctagactcctcaggagagacccctcccctcccacccacagccatagacctacagacctggctctcagaggaaaaatgggagccaggagtaagacaagtgggatttggggcctcaagaaatatactctcccagatttttactttttcccatctggctttttctgccactgaggagacagaaagcctccgctgggcttcattccggactggcagaagcattgcctggactgaccacaccaaccaggccttcatcctcctccccagctcttctcttcctagatctgcaggctgcacctctggctagagccgaggggagagagggactcaagggaaaggcaagcttgaggccaagatggctgctgcctgctcatggccctcggaggtccagctgggcctcctgcctccgggcaggcaaggtttacactgcggaagccaaaggcagctaagatagaaagctggactgaccaaagactgcagaacccccaggtggcctgcgtcttttttctcttcccttcccagaccaggaaaggcttggctggtgtatgcacagggtgtggtatgagggggtggttattggactccaggcctgaccagggggcccgaacagggacttgtttagagagcctgtcaccagagcttctctgggctgaatgtatgtcagtgctataaatgccagagccaacctggacttcctgtcattttcacaatcttggggctgatgaagaagggggtggggggagtttgtgttgttgttgctgctgtttgggttgttggtctgtgtaacatccaagccagagtttttaaagccttctggatccatggggggagaagtgatatggtgaagggaagtggggagtatttgaacacagttgaattttttctaaaaagaaaaagagataaatgagctttccaga

SEQ ID NO: 4 - DNA, 15728 bp, Artificial Sequence
  MUC2 cDNA sequence
caacccacaccgcccctgccagccaccatggggctgccactagcccgcctggcggctgtgtgcctggccctgtctttggcagggggctcggagctccagacagagggcagaacccgaaaccacggccacaacgtctgcagcacctggggcaacttccactacaagaccttcgacggggacgtcttccgcttccccggcctctgcgactacaacttcgcctccgactgccgaggctcctacaaggaatttgctgtgcacctgaagcggggtccgggccaggctgaggcccccgccggggtggagtccatcctgctgaccatcaaggatgacaccatctacctcacccgccacctggctgtgcttaacggggccgtggtcagcaccccgcactacagccccgggctgctcattgagaagagcgatgcctacaccaaagtctactcccgcgccggcctcaccctcatgtggaaccgggaggatgcactcatgctggagctggacactaagttccggaaccacacctgtggcctctgcggggactacaacggcctgcagagctattcagaattcctctctgacggcgtgctcttcagtcccctggagtttgggaacatgcagaagatcaaccagcccgatgtggtgtgtgaggatcccgaggaggaggtggcccccgcatcctgctccgagcaccgcgccgagtgtgagaggctgctgaccgccgaggccttcgcggactgtcaggacctggtgccgctggagccgtatctgcgcgcctgccagcaggaccgctgccggtgcccgggcggtgacacctgcgtctgcagcaccgtggccgagttctcccgccagtgctcccacgccggcggccggcccgggaactggaggaccgccacgctctgccccaagacctgccccgggaacctggtgtacctggagagcggctcgccctgcatggacacctgctcacacctggaggtgagcagcctgtgcgaggagcaccgcatggacggctgtttctgcccagaaggcaccgtatatgacgacatcggggacagtggctgcgttcctgtgagccagtgccactgcaggctgcacggacacctgtacacaccgggccaggagatcaccaatgactgcgagcagtgtgtctgtaacgctggccgctgggtgtgcaaagacctgccctgccccggcacctgtgccctggaaggcggctcccacatcaccaccttcgatgggaagacgtacaccttccacggggactgctactatgtcctggccaagggtgaccacaacgattcctacgctctcctgggcgagctggccccctgtggctccacagacaagcagacctgcctgaagacggtggtgctgctggctgacaagaagaagaatgtggtggtcttcaagtccgatggcagtgtactgctcaacgagctgcaggtgaacctgccccacgtgaccgcgagcttctctgtcttccgcccgtcttcctaccacatcatggtgagcatggccattggcgtccggctgcaggtgcagctggccccagtcatgcaactctttgtgacactggaccaggcctcccaggggcaggtgcagggcctctgcgggaacttcaacggcctggaaggtgacgacttcaagacggccagcgggctggtggaggccacgggggccggctttgccaacacctggaaggcacagtcaagctgccatgacaagctggactggttggacgatccctgctccctgaacatcgagagcgccaactacgccgagcactggtgctccctcctgaagaagacagagaccccctttggcaggtgccactcggctgtggaccctgctgagtattacaagaggtgcaaatatgacacgtgtaactgtcagaacaatgaggactgcctgtgcgccgccctgtcctcctacgcgcgcgcctgcaccgccaagggcgtcatgctgtggggctggcgggagcatgtctgcaacaaggatgtgggctcctgccccaactcgcaggtcttcctgtacaacctgaccacctgccagcagacctgccgctccctctccgaggccgacagccactgtctcgagggctttgcgcctgtggacggctgcggctgccctgaccacaccttcctggacgagaagggccgctgcgtacccctggccaagtgctcctgttaccaccgcggtctctacctggaggcgggggatgtggtcgtcaggcaggaagaacgatgtgtgtgccgggatgggcggctgcactgtaggcagatccggctgatcggccagagctgcacggccccaaagatccacatggactgcagcaacctgactgcactggccacctcgaagccccgagccctcagctgccagacgctggccgccggctattaccacacagagtgtgtcagtggctgtgtgtgccccgacgggctgatggatgacggccggggtggctgcgtggtggagaaggaatgcccttgcgtccataacaacgacctgtattcttccggcgccaagatcaaggtggactgcaatacctgcacctgcaagagaggacgctgggtgtgcacccaggctgtgtgccatggcacctgctccatttacgggagtggccactacatcacctttgatgggaagtactacgactttgacggacactgctcctacgtggctgttcaggactactgcggccagaactcctcactgggctcattcagcatcatcaccgagaacgtcccctgtggcactacgggcgtcacctgctccaaggccatcaagatcttcatggggaggacggagctgaagttggaagacaagcaccgtgtggtgatccagcgtgatgagggtcaccacgtggcctacaccacgcgggaggtgggccagtacctggtggtggagtccagcacgggcatcatcgtcatctgggacaagaggaccaccgtgttcatcaagctggctccctcctacaagggcaccgtgtgtggcctgtgtgggaactttgaccaccgctccaacaacgacttcaccacgcgggaccacatggtggtgagcagcgagctggacttcgggaacagctggaaggaggcccccacctgcccagatgtgagcaccaaccccgagccctgcagcctgaacccgcaccgccgctcctgggccgagaagcagtgcagcatcctcaaaagcagcgtgttcagcatctgccacagcaaggtggaccccaagcccttctacgaggcctgtgtgcacgactcgtgctcctgtgacacgggtggggactgtgagtgcttctgctctgccgtggcctcctacgcccaggagtgtaccaaagagggggcctgcgtgttctggaggacgccggacctgtgccccatattctgcgactactacaaccctccgcatgagtgtgagtggcactatgagccatgtgggaaccggagcttcgagacctgcaggaccatcaacggcatccactccaacatctccgtgtcctacctggagggctgctacccccggtgccccaaggacaggcccatctatgaggaggatctgaagaagtgtgtcactgcagacaagtgtggctgctatgtcgaggacacccactacccacctggagcatcggttcccaccgaggagacctgcaagtcctgcgtgtgtaccaactcctcccaagtcgtctgcaggccggaggaaggaaagattcttaaccagacccaggatggcgccttctgctactgggagatctgtggccccaacgggacggtggagaagcacttcaacatctgttccattacgacacgcccgtccaccctgaccaccttcaccaccatcaccctccccaccacccccaccaccttcaccactaccaccaccaccaccaccccgacctccagcacagttttatcaacaactccgaagctgtgctgcctctggtctgactggatcaatgaggaccaccccagcagtggcagcgacgacggtgaccgagaaacatttgatggggtctgcggggcccctgaggacatcgagtgcaggtcggtcaaggatccccacctcagcttggagcagctaggccagaaggtgcagtgtgatgtctctgttgggttcatttgcaagaatgaagaccagtttggaaatggaccatttggactgtgttacgactacaagatacgtgtcaattgttgctggcccatggataagtgtatcaccactcccagccctccaactaccactcccagccctccaccaaccagcacgaccacccttccaccaaccaccacccccagccctccaaccaccaccacaaccacccctccaccaaccaccacccccagccctccaataaccaccacgaccacccctccaccaaccaccactcccagccctccaataagcaccacaaccacccctccaccaaccaccactcccagccctccaaccaccactcccagccctccaaccaccactcccagccctccaacaaccaccacaaccacccctccaccaaccaccactcccagccctccaacgactacgcccatcactccaccagccagcactaccacccttccaccaaccaccactcccagccctccaacaaccaccacaaccacccctccaccaaccaccactcccagtcctccaacgactacgcccatcactccaccaaccagcactactacccttccaccaaccaccactcccagccctccaccaaccaccacaaccacccctccaccaaccaccactcccagccctccaacaaccaccactcccagtcctccaacaatcaccacaaccacccctccaccaaccaccactcccagccctccaacaacgaccacaaccacccctccaccaaccaccactcccagccctccaacgactacacccatcactccaccaaccagcactaccacccttccaccaaccaccactcccagccctccaccaaccaccacaaccacccctccaccaaccaccactcccagccctccaacaaccaccactcccagccctccaataaccaccacaaccacccctccaccaaccaccactcccagctctccaataaccaccactcccagccctccaacaaccaccatgaccaccccttcaccaaccaccacccccagctctccaataaccaccacaaccaccccttcctcaactaccactcccagccctccaccaaccaccatgaccaccccttcaccaaccaccactcccagccctccaacaaccaccatgaccacccttccaccaaccaccacttccagccctctaacaactactcctctacctccatcaataactcctcctacattttcaccattctcaacgacaacccctactaccccatgcgtgcctctctgcaattggactggctggctggattctggaaaacccaactttcacaaaccaggtggagacacagaattgattggagacgtctgtggaccaggctgggcagctaacatctcttgcagagccaccatgtatcctgatgttcccattggacagcttggacaaacagtggtgtgtgatgtctctgtggggctgatatgcaaaaatgaagaccaaaagccaggtggggtcatccctatggccttctgcctcaactacgagatcaacgttcagtgctgtgagtgtgtcacccaacccaccaccatgacaaccaccaccacagagaacccaactccgccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccccaaccacgacacccatcaccaccaccactacggtgaccccaaccccaacacccaccggcacacagaccgggccccccacccacacaagcacagcaccgattgctgagttgaccacatccaatcctccgcctgagtcctcaacccctcagacctctcggtccacctcttcccctctcacggagtcaaccacccttctgagtaccctaccacctgccattgagatgaccagcacggccccaccctccacacccacggcacccacgaccacgagcggaggccacacactgtctccaccgcccagcaccaccacgtcccctccaggcacccccactcgcggtaccacgaccgggtcatcttcagcccccacccccagcactgtgcagacgaccaccaccagtgcctggaccccaacgccgaccccactctccacacccagcatcatcaggaccacaggcctgaggccctacccttcctctgtgcttatctgctgtgtcctgaacgacacctactacgcaccaggtgaggaggtgtacaacggcacatacggagacacctgttatttcgtcaactgctcactgagctgtacgttggagttctataactggtcctgcccatccacgccctccccaacacccacgccctccaagtcgacgcccacgccttccaagccatcgtccacgccctccaagccgacgcccggcaccaagccccccgagtgcccagactttgatcctcccagacaggagaacgagacttggtggctgtgcgactgcttcatggccacgtgcaagtacaacaacacggtggagatcgtgaaggtggagtgtgagccgccgcccatgcccacctgctccaacggcctccaacccgtgcgcgtcgaggaccccgacggctgctgctggcactgggagtgcgactgctactgcacgggctggggcgacccgcactatgtcaccttcgacggactctactacagctaccagggcaactgcacctacgtgctggtggaggagatcagcccctccgtggacaacttcggagtttacatcgacaactaccactgcgatcccaacgacaaggtgtcctgtccccgcaccctcatcgtgcgccacgagacccaggaggtgctgatcaagaccgtgcatatgatgcccatgcaggtgcaggtgcaggtgaacaggcaggcggtggcactgccctacaagaagtacgggctggaggtgtaccagtctggcatcaactacgtggtggacatccccgagctgggtgtcctcgtctcctacaatggcctgtccttctccgtcaggctgccctaccaccggtttggcaacaacaccaagggccagtgtggcacctgcaccaacaccacctccgacgactgcattctgcccagcggggagatcgtctccaactgtgaggctgcggctgaccagtggctggtgaacgacccctccaagccacactgcccccacagcagctccacgaccaagcgcccggccgtcactgtgcccgggggcggtaaaacgaccccacacaaggactgcaccccatctcccctctgccagctcatcaaggacagcctgtttgcccagtgccacgcactggtgcccccgcagcactactacgatgcctgcgtgttcgacagctgcttcatgccgggctcgagcctggagtgcgccagtctgcaggcctacgcagccctctgtgcccagcagaacatctgcctcgactggcggaaccacacgcatggggcctgcttggtggagtgcccatctcacagggagtaccaggcctgtggccctgcagaagagcccacgtgcaaatccagctcctcccagcagaacaacacagtcctggtggaaggctgcttctgtcctgagggcaccatgaactacgctcctggctttgatgtctgcgtgaagacctgcggctgtgtgggacctgacaatgtgcccagagagtttggggagcacttcgagttcgactgcaagaactgtgtctgcctggagggtggaagtggcatcatctgccaacccaagaggtgcagccagaagcccgttacccactgcgtggaagacggcacctacctcgccacggaggtcaaccctgccgacacctgctgcaacattaccgtctgcaagtgcaacaccagcctgtgcaaagagaagccctccgtgtgcccgctgggattcgaagtgaagagcaagatggtgcctggaaggtgctgtcctttctactggtgtgagtccaagggggtgtgtgttcacgggaatgctgagtaccagcccggttctccagtttattcctccaagtgccaggactgcgtgtgcacggacaaggtggacaacaacaccctgctcaacgtcatcgcctgcacccacgtgccctgcaacacctcctgcagccctggcttcgaactcatggaggcccccggggagtgctgtaagaagtgtgaacagacgcactgtatcatcaaacggcccgacaaccagcacgtcatcctgaagcccggggacttcaagagcgacccgaagaacaactgcacattcttcagctgcgtgaagatccacaaccagctcatctcgtccgtctccaacatcacctgccccaactttgatgccagcatttgcatcccgggctccatcacattcatgcccaatggatgctgcaagacctgcacccctcgcaatgagaccagggtgccctgctccaccgtccccgtcaccacggaggtttcgtacgccggctgcaccaagaccgtcctcatgaatcattgctccgggtcctgcgggacatttgtcatgtactcggccaaggcccaggccctggaccacagctgctcctgctgcaaagaggagaaaaccagccagcgtgaggtggtcctgagctgccccaatggcggctcgctgacacacacctacacccacatcgagagctgccagtgccaggacaccgtctgcgggctccccaccggcacctcccgccgggcccggcgctcccctaggcatctggggagcgggtgagcggggtgggcacagcccccttcactgccctcgacagctttacctcccccggaccctctgagcctcctaagctcggcttcctctcttcagatatttattgtctgagtctttgttcagtccttgctttccaataataaactcagggggacatgctaaaaaaa

(19) United States
(12) Patent Application Publication
(10) Pub. No.: US 2011/0281277 A1
(43) Pub. Date: Nov. 17, 2011

(54) COMPOSITION, KIT, AND METHOD FOR DIAGNOSING COLORECTAL CANCER OR OVARIAN CANCER

(76) Inventor: Ah Won LEE, Seoul (KR)

(21) Appl. No.: 12/910,616

(22) Filed: Oct. 22, 2010

(30) Foreign Application Priority Data
May 17, 2010 (KR) 10-2010-0046155

(51) Int. Cl. C12Q 1/68; G01N 33/53; G01N 33/574; C07H 21/04; C07K 16/18

(52) U.S. Cl. 435/6.14; 536/24.33; 536/24.31; 530/389.1; 436/64; 435/7.1; 435/7.92

(57) ABSTRACT

The present invention relates to a composition for diagnosing colorectal cancer or ovarian cancer, comprising gene markers for quickly and correctly diagnosing colorectal cancer or ovarian cancer, and more particularly, to a composition for diagnosing colorectal cancer or ovarian cancer, comprising an agent measuring mRNA or protein levels of two or more genes selected from the group consisting of CK7 (cytokeratin 7), CK20 (cytokeratin 20), CDX2 (caudal type homeobox transcription factor 2), and MUC2 (mucin 2), a diagnostic kit comprising the composition, and a diagnostic method.

### TECHNICAL FIELD

The present invention relates to a composition, kit, and method for diagnosing colorectal cancer or ovarian cancer, and more particularly, to a composition, kit, and method for diagnosing colorectal cancer or ovarian cancer, which use CK7, CK20, CDX2, and MUC2 genes as diagnostic markers for colorectal cancer or ovarian cancer.

### BACKGROUND ART

At present, the incidence of colorectal cancer in South Korea is dramatically increasing, and deaths from colorectal cancer rank fourth after stomach cancer, lung cancer, and liver cancer for men and similar for women. It has been reported that the frequency of colorectal cancer is higher in men than women, and when analyzed by age, the frequency of colorectal cancer is the highest in people in their 50's, followed by people in their 60's.

It is currently believed that environmental factors may play a bigger role than genetic factors in the development of colorectal cancer. Rapid westernization of diet and excess intake of animal fat or protein are major factors in the development of colorectal cancer. However, it is known that about 5% of colorectal cancer cases occurs by a genetic cause.

With advances in science and technology, genes involved in the development of colorectal cancer have been identified, and methods for the early detection and treatment of colorectal cancer using these genes have been studied. It has been revealed that various genetic alterations, including other types of cancer genes, tumor suppressor genes, etc., are involved in colorectal cancer.

In reality, colorectal cancer is one of the cancers for which the largest number of genetic alterations occurring during carcinogenesis have been identified. A change in a single cancer gene or tumor suppressor gene alone cannot induce colorectal cancer, but changes in a number of cancer-related genes need to be accumulated for a long period of several years in order for normal large intestinal mucosal cells to develop into colorectal cancer through an intermediate adenoma stage, which is called multistep genetic alterations in the development of colorectal cancer. Such alterations reflect the total accumulation of genetic alterations, rather than the sequence of genetic alterations in each stage.

The results of continued studies of genes that cause colorectal cancer revealed that K-ras, APC, MCC, DCC, p53, and aberrant methylation of DNA is involved in genetic alterations associated with the development of colorectal cancer, and also revealed that mutations, such as hMSH2, hMSH1, hPMS1, hPMS2, etc., are associated with the development colorectal cancer. In this way, cancer formation occurs in complex association with various genes and the expression and regulation mechanisms of these genes. Therefore, in recent years, studies are being conducted to find new diagnoses of cancer or therapeutic markers by comparing the expression rate of cancer-related genes using an oligo chip containing a large number of genes.

In particular, genes whose expression is increased or decreased are involved in many processes, including cell division, cell signaling, cytoskeleton, cell movement, cell defense, expression of genes and proteins, intercellular metabolism, etc. Thus, while some genes show the same expression changes, other genes show different expression changes. Also, specificity in individual patients may be the cause different expression patterns, so cancer diagnosis should be/consistent with accurate pathological findings and classification of target patient tissue, and the diagnosis and identification of many genes are required for more accurate diagnosis.

The sensitivity and specificity of cancer diagnosis using one type of label are relatively lower than those of cancer diagnosis using many labels, thus resulting in a relatively high probability of error. There are some restrictions in order to use labels specific to identified cancer cells or tissues in early diagnosis. Firstly, the restriction on the concentration of a label will be described. If a label expressed in cancer tissue is released in body fluids such as plasma or spinal fluid, the label is excessively diluted, which makes it difficult to use the label as a diagnostic label. Therefore, it is hard to detect the label found in the tissue in the body fluids, and the significance of the label is considerably lessened in the case of investigation of the tissue itself. Moreover, there is a possibility that a material showing different expression in body fluids may not be consistent with a material that is altered in cancer or other diseased tissue. Further, changes in tissue may appear as completely different labels when they reach body fluids in a cascade manner, and even different cancer tissues may represent a common label. Accordingly, cancer diagnosis using a multiplex biomarker can serve as a measure for more accurate diagnosis, compared to cancer diagnosis using one type of marker.

Furthermore, 5 to 30% of ovarian cancer is metastatic malignancies. The prevalence of metastatic ovarian tumors differs among different countries according to the incidence of various cancers therein (1). In South Korea, colorectal cancer is one of the most common secondary cancers of the ovary (Bae J H, et al. J Korean Med Sci, 2009; 24: 114-9). Thus, the probability of misdiagnosis of colorectal cancer as ovarian cancer is high, and this may result in wrong therapies being administered to patients. Consequently, there is an urgent need for the development of new diagnostic methods for correctly diagnosing ovarian cancer and colorectal cancer.

### DISCLOSURE

### Technical Problem

Accordingly, it is an object of the present invention to provide a composition for diagnosing colorectal cancer or ovarian cancer, which uses CK7, CK20, CDX2, and MUC2 genes to quickly and correctly diagnose colorectal cancer or ovarian cancer.

It is another object of the present invention to provide a kit for diagnosing colorectal cancer or ovarian cancer, comprising the composition according to the present invention.

It is still another object of the present invention to provide a method for predicting and diagnosing colorectal cancer or ovarian cancer.

### Technical Solution

To accomplish the aforementioned objects, the present invention provides a composition for diagnosing colorectal cancer or ovarian cancer, comprising an agent measuring mRNA or protein levels of two or more genes selected from the group consisting of CK7, CK20, CDX2, and MUC2.

In one embodiment of the present invention, the composition may comprise an agent measuring mRNA or protein levels of a combination of genes selected from the group consisting of 1 to 8 listed in the following table:

| | gene combination |
|---|---|
| 1 | CK7/MUC2 |
| 2 | CK20/MUC2 |
| 3 | CDX2/MUC2 |
| 4 | CK7/CK20/MUC2 |
| 5 | CK7/CDX2/MUC2 |
| 6 | CK20/CDX2/MUC2 |
| 7 | CK7/CK20/CDX2/MUC2 |
| 8 | CK7/CK20/CDX2/CEA/MUC2/MUC5AC/AMACR |

In one embodiment of the present invention, the agent measuring mRNA levels of the genes may be a primer or probe specifically binding to a gene selected from the group consisting of CK7, CK20, CDX2, and MUC2.

In one embodiment of the present invention, the agent measuring protein levels may be an antibody specific to a protein selected from the group consisting of CK7, CK20, CDX2, and MUC2.

Moreover, the present invention provides a kit for diagnosing colorectal cancer or ovarian cancer, comprising the composition according to the present invention.

In one embodiment of the present invention, the kit may be an RT-PCR kit, a DNA chip kit, or a protein chip kit.

Further, the present invention provides a method for predicting and diagnosing colorectal cancer or ovarian cancer, comprising the steps of: (a) measuring expression of two or more genes or proteins selected from the group consisting of CK7, CK20, CDX2, and MUC2 present in a biological sample; and (b) comparing the measurement result in the step (a) with the expression of genes or proteins of normal control samples.

In one embodiment of the present invention, the biological sample may be selected from the group consisting of tissues, cells, whole blood, serum, plasma, saliva, and urine.

In one embodiment of the present invention, the measurement may be selected from the group consisting of reverse transcriptase-polymerase chain reaction, real time-polymerase chain reaction, Western blot, Northern blot, ELISA (enzyme linked immunosorbent assay), RIA (radioimmunoassay), radioimmunodiffusion, immunohistochemistry, and immunoprecipitation assay.

In one embodiment of the present invention, if the expression of the CK7 gene or protein is increased compared to the normal control samples and the expression of the CK20, CDX2, and MUC2 genes or proteins is decreased compared to the normal control samples, the onset of ovarian cancer is determined.

In one embodiment of the present invention, if the expression of the CK7 gene or protein is decreased compared to the normal control samples and the expression of the CK20, CDX2, and MUC2 genes or proteins is increased compared to the normal control samples, the onset of colorectal cancer is determined.

### Advantageous Effects

The CK7, CK20, CDX2, and MUC2 genes according to the present invention have different expression patterns in colorectal cancer and ovarian cancer. Therefore, these genes can be used as diagnostic markers for distinguishing colorectal cancer from ovarian cancer, and a multiplex marker of two or more gene combinations of the genes is able to correctly diagnose colorectal cancer and ovarian cancer at an early stage by distinguishing them from each other, thus improving the reliability of diagnosis. This enables quick treatment and improves the survival rates of patients with colorectal cancer or ovarian cancer.

### DESCRIPTION OF DRAWINGS

The above and other objects and features of the present invention will become apparent from the following description of the preferred embodiments given in conjunction with the accompanying drawings, in which:

FIG. 1 shows photographs showing expression levels of the CK7, CK20, CDX2, and MUC2 genes observed in primary ovarian mucinous adenocarcinoma (POMA) tissue through immunohistochemistry, which depict results of observation using A:CK7, B:CK20, C:CDX2, and D:MUC2 antibodies; and

FIG. 2 shows photographs showing expression levels of the CK7, CK20, CDX2, and MUC2 genes observed in metastatic colorectal adenocarcinoma (MCAO) tissue through immunohistochemistry, which depict results of observation using A:CK7, B:CK20, C:CDX2, and D:MUC2 antibodies.

### BEST MODE FOR THE INVENTION

The present invention provides biomarkers for quickly and correctly diagnosing colorectal cancer or ovarian cancer at an early stage by distinguishing them from each other, and more particularly, to a composition for diagnosing colorectal cancer or ovarian cancer, comprising an agent measuring mRNA or protein levels of two or more genes selected from the group consisting of CK7, CK20, CDX2, and MUC2.

The present inventors paid attention to CK7 (cytokeratin 7), CK20 (cytokeratin 20), CDX2 (caudal type homeobox transcription factor 2), and MUC2 (mucin 2) genes in order to develop novel diagnostic markers for correctly diagnosing colorectal cancer and ovarian cancer by distinguishing them from each other. The CK7 (cytokeratin 7) and CK20 (cytokeratin 20) genes belonging to the cytokeratin family are antigen proteins that are expressed on the surface of most epithelial cells. The expression of such proteins indicates the presence of epithelial cells in blood. In particular, cytokeratin proteins are expressed in cancer cells originating from the epithelial cells, so CK7 (cytokeratin 7) and CK20 (cytokeratin 20) are used to diagnose cancer originating from the epithelial cells.

Moreover, the CDX2 (caudal type homeobox transcription factor 2), a homologue of the caudal gene found first in drosophila, encodes a homeobox transcription factor. It is known that the CDX2 gene is specifically expressed in epithelial cells of the small and large intestines of a human body, plays an important role in the proliferation and differentiation of normal epithelial cells, and functions as a tumor suppressor gene. Also, as a recent study has shown that CDX2 expression is lost in colorectal cancer cells, CDX2 is used as a diagnostic marker for colorectal cancer.

The MUC2 gene, one of the mucin genes, is a gene belonging to secretary mucin. Mucin is a major component of mucus, which functions to secrete mucus to the epithelial cells of the digestive system, and respiratory organs such as the airway. Thus, it functions to protect the intestinal surface, which is epithelial tissue, from the mechanical damage and chemical stimulation of each organ and acts as a lubricant for a bowel movement. 20 mucin genes performing such functions have been identified to date and can be broadly divided, according to function, into secretory mucins and membrane-bound mucins. In particular, when the secretory mucin genes, to which the MUC2 gene belongs, are in a normal state, they will secrete mucus from different organs to protect each organ and the intestines, but when they are regulated or have abnormality, they will excessively secrete mucus. It has been reported that, in the case of bronchi, such excessive secretion causes asthma or involves inflammatory disease, and in the case of gastric cancer, excessive mucus increases tolerance to various pathogenic bacteria, thus increasing the incidence of gastric cancer. However, the use of the MUC2 gene as a diagnostic marker for colorectal cancer or ovarian cancer has not been reported yet.

As discussed above, in the present invention, the CK7, CK20, CDX2, and MUC2 genes used as diagnostic biomarkers for distinguishing colorectal cancer from ovarian cancer can be used as markers for cancer diagnosis because the expression levels of these genes in some particular cancer cells are different from those in normal cells. However, since various genes are involved in carcinogenesis, the use of one type of label may lead to the problem of a high probability of error due to low sensitivity and specificity.

Hereupon, the present invention has clarified for the first time that, when a multiplex marker including two or more of the CK7, CK20, CDX2, and MUC2 genes is used as a diagnostic biomarker for distinguishing colorectal cancer from ovarian cancer in order to improve the low sensitivity and specificity of conventional cancer diagnostic markers, colorectal cancer or ovarian cancer can be diagnosed with a high accuracy.

In general, one of the most common metastatic cancers associated with the ovary is colorectal cancer. Colorectal cancer mimics the symptoms of primary ovarian carcinoma, and colorectal cancer is frequently misdiagnosed as ovarian cancer. This misdiagnosis leads to treatments for ovarian cancer, instead of treatments for colorectal cancer, ultimately increasing the pain of patients.

Accordingly, at this point in time when there is a need for a novel diagnostic method for correctly distinguishing colorectal cancer from ovarian cancer at an early stage, the present inventors have found that, when a multiplex biomarker of two or more of the CK7, CK20, CDX2, and MUC2 genes is used, colorectal cancer and ovarian cancer can be distinguished and diagnosed correctly.

That is, according to one embodiment of the present invention, the present inventors obtained cancer tissue from patients with POMA (primary ovarian mucinous adenocarcinoma and MCAO (metastatic colorectal adenocarcinoma) and then constructed tissue array blocks. The inventors performed immunohistochemistry using an antibody for detecting the expression of primarily selected candidate marker genes to compare and analyze the expression levels of the candidate marker genes.

As a result of comparison and analysis of the expression of the CK7, CK20, CDX2, CEA, MUC2, MUC5AC and AMACR genes, POMAs were almost negative for the MUC2 and CDX2 genes, focal positive for CK20 and CEA, and positive for CK7 and MUC5AC. MCAOs were negative for MUC5AC, negative for CK7, focal positive for CDX2 and MUC2, and diffuse positive (i.e., overexpression) for CK20 and CEA (see Example 1).

From the above result, the present inventors have found that the CK7, CK20, CDX2, CEA, MUC2, MUC5AC and AMACR genes showing different expression patterns in colorectal cancer and ovarian cancer can be used as markers for predicting or diagnosing the onset of these cancers.

Moreover, according to another embodiment of the present invention, the sensitivity, specificity, positive predictive value, and negative predictive value of the CK7, CK20, CDX2, CEA (carcinoembryonic antigen), MUC2, MUC5AC (mucin 5 subtype A and C), and AMACR (α-methlacyl-CoA racemase) genes showing different expression patterns in colorectal cancer and ovarian cancer were calculated by statistical calculation. The calculation demonstrated that the CK7, CK20, CDX2, and MUC2 genes showed high values for the above properties (see Example 2).

Hereupon, in order to find out whether colorectal cancer and ovarian cancer can be distinguished and diagnosed using the selected CK2, CK20, CDX2, and MUC2 genes, the present inventors performed immunohistochemstry on colorectal cancer tissue and ovarian cancer tissue using an antibody against the four proteins and analyzed the expression pattern of the proteins in each tissue. As a result, POMAs were positive for CK7 and negative for CK20, CDX2, and MUC2 (i.e., almost no protein expression). On the contrary to the result of POMAs, MCAOs were negative for CK7 and positive for CK20, CDX2, and MUC2.

By this, the present inventors have found that, when the CK7, CK20, CDX2, and MUC2 genes are used, colorectal cancer and ovarian cancer can be correctly distinguished and diagnosed while reducing the incidence of misdiagnosis of colorectal cancer and ovarian cancer, which has conventionally been a problem, by a simple method of assessing the expression levels of the genes.

Furthermore, while devising a method for enhancing the diagnostic rate of colorectal cancer and ovarian cancer using the genes according to the present invention, the present inventors have found that, when a multiplex biomarker of two or more of the CK7, CK20, CDX2, and MUC2 genes is used, the diagnostic rate can be improved.

That is, according to one embodiment of the present invention, as a result of analysis of the diagnosis accuracy of ovarian cancer and colorectal cancer using a multiplex marker consisting of two genes among the four genes, a multiplex marker consisting of three genes among the four genes, and a multiplex marker consisting of the four genes, the use of two genes showed an accuracy of about 50 to 70%, the use of three genes showed an accuracy of 70 to 85%, and the use of all of the four genes showed a high accuracy of 87.3%.

Also, when all of the seven genes, including CEA, MUC5AC, and AMACR that showed different expression patterns in colorectal cancer and ovarian cancer, in addition to the above four genes, were used, a high accuracy of 74.6% was obtained.

Accordingly, from this result, the present inventors have found that, when a combination of two or more of the CK7, CK20, CDX2, and MUC2 genes according to the present invention is used as a multiplex marker, colorectal cancer or ovarian cancer can be quite correctly diagnosed.

Therefore, putting the above results together, the present invention provides diagnostic markers for colorectal cancer or ovarian cancer, comprising two or more genes selected from the group consisting of CK7, CK20, CDX2, and MUC2 or proteins expressed from the genes.

In the context of the present invention, the term “diagnosis” refers to the detection of a pathological state.

For the purpose of the invention, the diagnosis is to confirm the development of colorectal cancer or ovarian cancer by assessing the expression of diagnostic markers for colorectal cancer or ovarian cancer. In addition, in the context of the present invention, the “diagnosis” includes determining the onset, progression, or amelioration of colorectal cancer or ovarian cancer by assessing the expression or lack of expression of diagnostic markers for colorectal cancer or ovarian cancer and the expression levels thereof.

The term “a diagnostic marker, a marker for diagnosis, or a diagnosis marker”, as used herein, is intended to indicate a substance that can diagnose colorectal cancer cells or ovarian cancer by distinguishing colorectal cancer cells or ovarian cancer cells from normal cells, and includes organic biological molecules, quantities of which increase or decrease in colorectal cancer cells or ovarian cancer cells compared to normal cells, such as polypeptides or nucleic acids (e.g., mRNA, etc.), lipids, glycolipids, glycoproteins, and sugars (monosaccharides, disaccharides, oligosaccharides, etc.). The diagnostic markers for colorectal cancer or ovarian cancer provided in the present invention, which are genes or proteins showing different expression patterns in colorectal cancer or ovarian cancer compared to normal cells, may be two or more genes or proteins selected from the group consisting of CK7, CK20, CDX2, and MUC2. Preferably, CK7 may have a base sequence of SEQ ID No. 1, CK20 may have a base sequence of SEQ ID No. 2, CDX2 may have a base sequence of SEQ ID No. 3, and MUC2 may have a base sequence of SEQ ID No. 4.

The selection and application of significant diagnostic markers for diagnosing diseases are factors that determine the reliability of diagnosis results. A “significant diagnostic marker” means a marker that is highly valid by making an accurate diagnosis and is highly reliable by providing constant results upon repeated measurement.

The diagnostic markers for colorectal cancer or ovarian cancer of the present invention, which are genes whose expression increases due to direct or indirect factors when colorectal cancer or ovarian cancer develops, display the same results upon repeated tests, and have high reliability due to a great difference in expression levels compared to a control, thus having a very low possibility of giving false results. Therefore, a diagnosis based on the results obtained by measuring the expression levels of the significant diagnostic markers of the present invention is valid and reliable.

Moreover, the present invention provides a composition for diagnosing colorectal cancer or ovarian cancer, comprising an agent measuring the levels of two or more genes or proteins selected from the group consisting of CK7, CK20, CDX2, and MUC2.

In the present invention, the composition for diagnosing colorectal cancer or ovarian cancer may preferably comprise an agent measuring mRNA or protein levels of a combination of genes selected from the group consisting of 1 to 8 listed in the following table, and the most preferable combination of genes may be a combination of the CK7, CK20, CDX2, and MUC2 genes.

TABLE 1
Combinations of Genes for Diagnosing
Colorectal Cancer or Ovarian Cancer

| | gene combination |
|---|---|
| 1 | CK7/MUC2 |
| 2 | CK20/MUC2 |
| 3 | CDX2/MUC2 |
| 4 | CK7/CK20/MUC2 |
| 5 | CK7/CDX2/MUC2 |
| 6 | CK20/CDX2/MUC2 |
| 7 | CK7/CK20/CDX2/MUC2 |
| 8 | CK7/CK20/CDX2/CEA/MUC2/MUC5AC/AMACR |

In the present invention, the levels of genes preferably refer to mRNA levels, i.e., amounts of mRNA, expressed from two or more genes selected from the group consisting of CK7, CK20, CDX2, and MUC2, and the agent for measuring the levels may comprise a primer or probe specific to the CK7, CK20, CDX2, and MUC2 genes.

In the present invention, the primer or probe specific to the CK7, CK20, CDX2, and MUC2 genes may be a primer or probe capable of specifically amplifying the entire regions of the CK7, CK20, CDX2, and MUC2 genes or specific regions of the genes, and the primer or probe can be designed by methods known in the art.

In the present invention, the “primer” as used herein refers to a single-strand oligonucleotide capable of initiating a template-directed DNA synthesis in an appropriate buffer under an appropriate condition (for example, in the presence of four different nucleoside triphosphates and a polymerizing agent such as DNA, RNA polymerase or reverse transcriptase) at a proper temperature. The length of the primer may vary according to various factors, for example, temperature and the use of the primer. The sequence of the primer is not required to be perfectly complementary to a part of the sequence of the template. The primer may have sufficient complementarity to be hybridized with the template and perform intrinsic functions of the primer. Thus, the primer of the present invention is not required to be perfectly complementary to the nucleotide sequence of the CK7, CK20, CDX2, or MUC2 gene used as a template. The primer of the present invention may have sufficient complementarity to be hybridized with the sequence of the gene and perform the functions of the primer. Moreover, the primer of the present invention is preferably used for gene amplification.

The “amplification” refers to nucleic acid amplification. Examples of amplification techniques of genes are known in the art, and may include polymerase chain reaction (PCR), reverse-transcription polymerase chain reaction (RT-PCR), ligase chain reaction (LCR), transcription-mediated amplification (TMA), nucleic acid sequence-based amplification (NASBA), etc.

In the present invention, the term “probe” as used herein refers to a linear oligomer of natural or modified monomers or linkages, including deoxyribonucleotides, ribonucleotides and the like, capable of specifically hybridizing with a target nucleotide sequence, whether occurring naturally or produced synthetically. The probe of the present invention may be single stranded, preferably, an oligodeoxyribonucleotide. The probe of this invention can be comprised of naturally occurring dNMP (i.e., dAMP, dGMP, dCMP and dTMP), nucleotide analogs, or nucleotide derivatives. The probe of this invention can also include ribonucleotides. For example, the probe of this invention may include nucleotides with backbone Modifications such as peptide (nucleic acid (PNA) (M. Egholm et al., Nature, 365:566-568 (1993)), phosphorothioate DNA, phosphorodithioate DNA, phosphoramidate DNA, amide-linked DNA, MMI-linked DNA, 2′-O-methyl RNA, alpha-DNA and methylphosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, pyridyl-), inosine, and diaminopurine.

In the present invention, examples of the agent for measuring protein levels may include “antibodies” including polyclonal antibodies, monoclonal antibodies, and recombinant antibodies that may specifically bind to CK7, CK20, CDX2, and MUC2 proteins.

In the present invention, antibody production using the CK2, CK20, CDX2, and MUC2 proteins as marker proteins for diagnosing colorectal cancer or ovarian cancer identified as described above may be easily carried out using techniques widely known in the art. For example, polyclonal antibodies may be produced using a method widely known in the art, which includes injecting a CK7, CK20, CDX2, or MUC2 antigen into an animal and collecting blood samples from the animal to obtain sera containing antibodies. Such polyclonal antibodies may be prepared from a certain animal host, such as goats, rabbits, sheep, monkeys, horses, pigs, cows and dogs. Monoclonal antibodies may be prepared by a method widely known in the art, such as a hybridoma method (see, Kohler and Milstein (1976) European Journal of Immunology 6:511-519), or a phage antibody library technique (Clackson et al., Nature, 352:624-628, 1991; Marks et al., J. Mol. Biol., 222:58, 1-597, 1991).

The antibodies of the present invention include complete forms, each of which consist of two full-length light chains and two full-length heavy chains, as well as functional fragments of antibody molecules. The functional fragments of antibody molecules refer to fragments retaining at least an antigen-binding function, and include Fab, F(ab′), F(ab′)2 and Fv.

Further, the present invention provides a kit for diagnosing colorectal cancer or ovarian cancer, comprising the composition for diagnosing colorectal cancer or ovarian cancer according to the present invention.

The composition for diagnosing colorectal cancer or ovarian cancer included in the kit for diagnosing colorectal cancer or ovarian cancer of the present invention may comprise an agent measuring mRNA or protein levels of two or more genes selected from the group consisting of CK7, CK20, CDX2, and MUC2 as described above. The agent for measuring mRNA of the genes may comprise a primer or probe, and the agent for measuring proteins may comprise an antibody, the definitions of which were described previously.

If the kit for diagnosing colorectal cancer or ovarian cancer of the present invention is used in the PCR amplification procedure, the kit of the present invention may selectively include reagents required for PCR amplification, for example, buffer, DNA polymerase (for example, thermostable DNA polymerase obtained from Thermus aquaticus (Taq), Thermus thermophilus (Tth), Thermus filiformis, Thermis flavus, Thermococcus literalis or Pyrococcus furiosus (Pfu)), DNA co-polymerase and dNTPs. If the diagnostic kit for colorectal cancer or ovarian cancer of the present invention is applied to immunoassay, the kit of the present invention may selectively comprise a secondary antibody and a labeled substrate.

Further, the kit of the present invention may be made of a plurality of packagings or compartments including the above reagent components. The type of kit to be produced in the present invention may be an RT-PCR kit, a DNA chip kit, or a protein chip kit, but not limited thereto.

In addition, the present invention provides a diagnostic microarray for colorectal cancer or ovarian cancer, comprising the composition for diagnosing colorectal cancer or ovarian cancer according to the present invention.

In the microarray of the present invention, the primers, probes, or antibodies for measuring CK7, CK20, CDX2, and MUC2 proteins or expression levels of genes encoding these proteins serve as hybridizable array elements and are immobilized on substrates. A preferable substrate includes suitable solid or semi-solid supporters, such as membrane, filter, chip, slide, wafer, fiber, magnetic or nonmagnetic bead, gel, tubing, plate, polymer, microparticle and capillary tube. The hybridizable array elements are arranged and immobilized on the substrate. Such immobilization occurs through chemical binding or covalent binding such as UV. In an example, the hybridizable array elements are bound to a glass surface modified to contain epoxy compound or aldehyde group or to a polylysine-coated surface by UV irradiation. Further, the hybridizable array elements are bound to a substrate through linkers (e.g. ethylene glycol oligomer and diamine).

If samples to be applied to the microarray of this invention are nucleic acids, they may be labeled, and hybridized with array elements on microarray. Various hybridization conditions are applicable, and for the detection and analysis of the extent of hybridization, various methods are available depending on labels used.

Further, the present invention provides a method for predicting and diagnosing colorectal cancer or ovarian cancer by measuring the expression levels of the marker genes for diagnosing colorectal cancer or ovarian cancer according to the present invention. Preferably, the method may comprise the steps of: (a) measuring expression of two or more genes or proteins selected from the group consisting of CK7, CK20, CDX2, and MUC2 present in a biological sample; and (b) comparing the measurement result in the step (a) with the expression of genes or proteins of normal control samples.

The aforementioned method for measuring the levels of the marker genes for diagnosing colorectal cancer or ovarian cancer, that is, two or more genes or proteins selected from the group consisting of CK7, CK20, CDX2, and MUC2 may be carried out by including a known process for isolating mRNA or protein from a biological sample by using the known art.

In the present invention, the term “biological sample”, as used herein, refers to samples derived from living organisms which show a difference in the expression levels of two or more genes or proteins selected from the group consisting of CK7, CK20, CDX2, and MUC2 from a normal control group depending on the degree of the onset or progression of colorectal cancer or ovarian cancer, and the samples may include tissues, cells, whole blood, serum, plasma, saliva, or urine, but are not limited thereto.

Preferably, the measurement of the expression levels of two or more genes selected from the group consisting of CK7, CK20, CDX2, and MUC2 involves measuring mRNA levels. Methods for measuring mRNA levels include reverse transcriptase-polymerase chain reaction (RT-PCR), real time reverse transcriptase-polymerase chain reaction, RNase protection analysis, Northern blotting, DNA chip, etc, but are not limited thereto.

The measurement of the levels of two or more proteins selected from the group consisting of CK7, CK20, CDX2, and MUC2 can be carried out using an antibody. In this case, CK7, CK20, CDX2, and MUC2 proteins in a biological sample and an antibody specific thereto form complexes, i.e., antigen-antibody complexes. The amount of formed antigen-antibody complexes may be quantitatively determined by measuring the signal size of a detection label. Such a detection label may be selected from the group consisting of enzymes, fluorescent substances, ligands, luminescent substances, microparticles, redox molecules and radioactive isotopes, but the present invention is not limited to the examples. Analysis methods for measuring protein levels include, but are not limited to, Western blotting, ELISA, radioimmunoassay, radioimmunodiffusion, ouchterlony immunodiffusion, rocket immunoelectrophoresis, immunohistochemistry, immunoprecipitation assay, complement fixation assay, FACS, and protein chip assay.

Accordingly, by the detection methods of the present invention, the expression of mRNA or proteins of two or more genes selected from the group consisting of CK7, CK20, CDX2, and MUC2 in normal control samples and the expression of mRNA or proteins of two or more genes selected from the group consisting of CK7, CK20, CDX2, and MUC2 in patients with colorectal cancer or ovarian cancer or patients suspected of having the cancers can be assessed. The onset, progression, or prognosis of colorectal cancer or ovarian cancer can be predicted and diagnosed by comparing the level of expression with that of the control group.

Particularly, the method for predicting and diagnosing colorectal cancer or ovarian cancer according to the present invention is characterized in that: colorectal cancer and ovarian cancer can be distinguished and diagnosed using the diagnostic markers according to the present invention. That is, if the expression of the CK7 gene or protein, among the CK7, CK20, CD2, and MUC2 genes, is increased compared to the normal control samples and the expression of the CK20, CDX2, and MUC2 genes or proteins is decreased compared to the normal control samples, the onset of ovarian cancer is determined.

In one embodiment of the present invention, if the expression of the CK7 gene or protein is decreased compared to the normal control samples and the expression of the CK20, CDX2, and MUC2 genes or proteins is increased compared to the normal control samples, the onset of colorectal cancer is determined.

Hereinafter, the present invention will be described in detail with respect to Examples. However, these Examples are intended to describe the present invention in further detail and should not be construed as limiting the scope of the invention.

### Example 1

Analysis of Expression of Genes Specific for Colorectal Cancer or Ovarian Cancer

<1-1> Obtaining of Samples

22 samples of POMA and 44 samples of MCAO with mucinous differentiation involving ovary were obtained between January 1996 and July 2008 at Seoul St Mary's Hospital. Mucinous differentiation was defined by the presence of at least focal areas of intracytoplasmic mucin globules in the tumor cells. The samples were reviewed and reclassified according to the 2003 WHO guidelines. Borderline mucinous tumors and microinvasive mucinous adenocarcinomas were excluded. The mean age of patients from whom the tumor samples were obtained was 42 years. The mean age of patients with metastatic mucinous adenocarcinoma from colorectum was 47 years. All but one of the samples of primary mucinous adenocarcinoma were surgically staged. Stages included: Stage I in 13 samples, Stage II in 2 samples, Stage III in 5 samples and Stage IV in 1 sample. Of the 22 patients with POMA, 19 patients were treated with surgery and adjuvant chemotherapy and 3 patients were treated with surgery only.

<1-2> Tissue Array

A tissue microarray block was constructed using the cancer tissue samples obtained in <1-1>. That is, 3 mm core biopsies were taken from paraffin embedded tumor tissues and assembled on a recipient paraffin block. This was carried out using a precision instrument (Micro Digital Co., Gunpo-si, Gyeonggi-do, Korea). After construction, 4 μm sections were cut and histology was verified by hematoxylin-eosin staining.

<1-3> Immunohistochemistry

Four-micrometer sections of the paraffin-embedded tissue arrays taken in <1-2> were deparaffinized, rehydrated in alcohol and microwave-treated for 10 min in a citrate buffer (pH 6.0). Endogenous peroxidase activity was blocked using 0.3% hydrogen peroxide. The tissue arrays were processed in an automatic IHC staining machine using the standard protocols with DAKO ChemMate™ EnVision™ system (DAKO, Carpinteria, Calif., USA). The following antibodies were used: cytokeratin 7 (CK7) (1:50, OV-TL 12/30, DAKO), cytokeratin 20 (CK20) (1:50, Ks20.8, DAKO), CEA (1:50, 11-7, DAKO), CDX2 (1:100, CDX2-88, BioGenex, San Ramon, Calif., USA), MUC2 (1:100, Ccp58, Novocastra, Newcastle, UK), MUC5AC (1:100, CLH2, Novocastra) and α-methylacyl-CoA racemase (AMACR) (1:100, rabbit polyclonal, Biocare Medical, Walnut Creek, Calif., USA). The sections were visualized with 3-3′-diaminobenzidine and tissue arrays were counterstained with Mayer's hematoxylin.

CK7, CK20, CDX2, CEA, MUC2, MUC5AC and AMACR expression in each tissue were classified based on the fraction of tumor cells showing positive cytoplasmic staining (negative, 1-10%; focal positive, 11-50%; and diffuse positive, ≧50%).

All statistical analyses performed in this example were performed using SPSS version 15.0 (Systat, Chicago, Ill., USA) for windows. A χ² test was used to compare the IHC results of POMAs and MCAOs. Survival duration was defined as the time from surgery to death. Survival curves were plotted using the Kaplan and Meier method and statistical significance was determined by the log-rank test. A P value <0.05 was considered significant. The results were shown in the following Table 2.

TABLE 2

| | Primary cancer (n = 22) | | | | | | Colonic cancer (n = 41) | | | | | |
|---|---|---|---|---|---|---|---|---|---|---|---|---|
| | Negative (%) | | Focal+ (%) | | Diffuse+ (%) | | Negative (%) | | Focal+ (%) | | Diffuse+ (%) | |
| CK7 | 2 | (9.1) | 2 | (9.1) | 18 | (81.8) | 34 | (82.9) | 3 | (7.3) | 4 | (9.8) |
| CK20 | 12 | (54.5) | 8 | (36.4) | 2 | (9.1) | 10 | (24.4) | 4 | (9.8) | 27 | (65.9) |
| CDX2 | 20 | (90.9) | 1 | (4.5) | 1 | (4.5) | 11 | (26.8) | 4 | (9.8) | 26 | (63.4) |
| CEA | 17 | (77.3) | 4 | (18.2) | 1 | (4.5) | 13 | (31.7) | 11 | (26.8) | 17 | (41.5) |
| MUC2 | 22 | (100) | 0 | (0) | 0 | (0) | 20 | (48.8) | 10 | (24.4) | 11 | (26.8) |
| MUC5AC | 11 | (50) | 1 | (4.5) | 10 | (45.5) | 40 | (97.6) | 1 | (2.4) | 0 | (0) |
| AMACR | 18 | (81.8) | 3 | (13.6) | 1 | (4.5) | 18 | (41.5) | 8 | (19.5) | 15 | (36.6) |

AMACR, α-methylacyl-CoA racemase

The expression of CK7, CK20, CDX2, CEA, MUC2, MUC5AC and AMACR genes in the cancer tissues through immunohistochemistry on the tissues samples of POMAs and MCAOs was shown in Table 2.

POMAs were almost negative for MUC2 (100% negative), focal positive for CK20 (54.5% negative) and CEA (77.3% negative), and positive for CK7 (9.1% negative) and MUC5AC (50% negative). MCAOs were always negative for MUC5AC (97.6& negative), generally negative for CK7 (82.9% negative), focal positive for CDX2 (26.8% negative and 73.2% positive) and MUC2 (48.8% negative and 51.2% positive), and diffuse positive for CK20 (24.4% negative and 75.6% positive) and CEA (41.5% positive).

From the above result, the present inventors have found that, when the tissue samples obtained from the patients were negative for the CK7 and MUC5AC genes, diffuse positive for CK20 and CEA, and positive for CDX2, MUC2, and AMACR genes, a diagnosis of colorectal cancer can be made. Further, they have found that colorectal cancer and ovarian cancer can be distinguished and diagnosed through the analysis of expression patterns of the genes.

### Example 2

Selection of Genes Showing Colorectal Cancer-Specific Patterns Different from Ovarian Cancer

Further, the present inventors selected genes showing MCAO-specific expression patterns different from the expression pattern of POMA based on the above results. The sensitivity, specificity, positive predictive value and negative predictive value of these selected genes are shown in the following Table 3 through statistical calculations.

TABLE 3

| | Sensitivity (%) | Specificity (%) | PPV (%) | NVP (%) |
|---|---|---|---|---|
| CK7 | 0.83 | 0.91 | 0.94 | 0.74 |
| CK20 | 0.66 | 0.91 | 0.93 | 0.59 |
| CDX2 | 0.73 | 0.91 | 0.94 | 0.65 |
| CEA | 0.41 | 0.95 | 0.94 | 0.47 |
| MUC2 | 0.51 | 1.00 | 1.00 | 0.52 |
| MUC5AC | 0.98 | 0.5 | 0.78 | 0.92 |
| AMACR | 0.56 | 0.82 | 0.85 | 0.5 |

PPV. positive predictive value: NPV. negative predictive value.

As a result, as shown in Table 3, the CK7, CK20, CDX2, and MUC2 genes, among the genes showing colorectal cancer-specific expression patterns, demonstrate excellent sensitivity and specificity and high predictive values of expression in each colorectal cancer tissue. Thus, it was found that the genes can be used as markers for diagnosing colorectal cancer by distinguishing it from ovarian cancer.

### Example 3

Analysis of Expression of Genes Showing Colorectal Cancer-Specific Expression Patterns in Colorectal Cancer and Ovarian Cancer

To compare the expression patterns of the CK7, CK20, CX2, and MUC2 genes, which are colorectal cancer-specific genes selected in Example 2, in MCAO tissue and POMA tissue, immunohistochemical staining was performed using antibodies for detecting the expression of the genes, i.e., antibodies against the CK7, CK20, CDX2, and MUC2 used in Example 1.

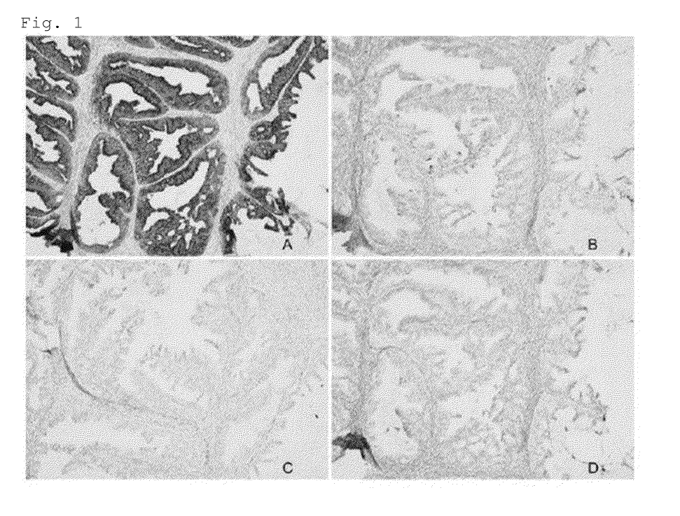

As a result, as shown in FIG. 1, POMAs were diffuse negative for CK7, and negative for CK20, CDX2, and MUC2 (i.e., almost no protein expression).

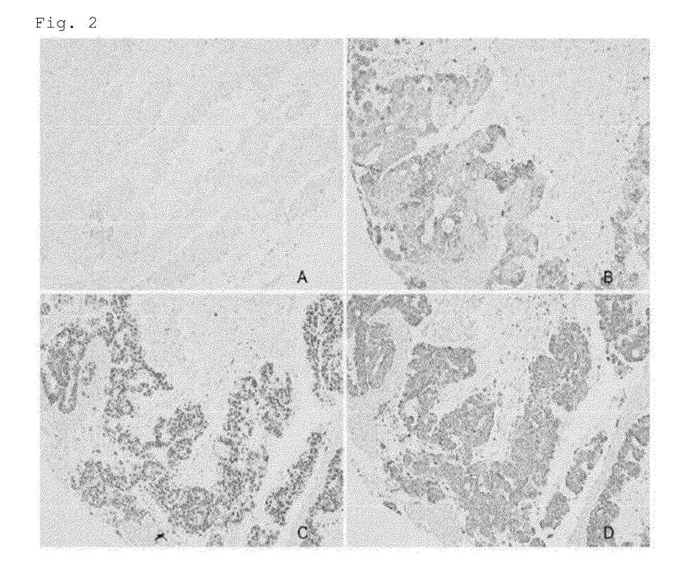

On the contrary, as shown in FIG. 2, MCAOs were negative for CK7 and positive for CK20, CDX2, and MUC2.

Accordingly, from the above result, the present inventors have found that, when the CK7, CK20, CDX2, and MUC2 genes selected in the present invention can be used as diagnostic markers for distinguishing ovarian cancer from colorectal cancer. In particular, if the CK20, CDX2, and MUC2 genes are simultaneously expressed and the CK7 gene is not expressed, it can be found that the onset of colorectal cancer or the progression of metastasis of colorectal cancer can be predicted or diagnosed.

### Example 4

Analysis of Cancer Diagnostic Rate Using Combinations of Selected Genes

Further, the present inventors investigated gene combinations for most correctly diagnosing the level of the onset of colorectal cancer by analyzing the diagnostic rate for each combination when the CK7, CK20, CDX2, and MUC2 genes for diagnosing colorectal cancer selected in the present invention are used as markers. To this end, cancer samples were taken from a total of 63 cancer tissues obtained in Example 1, and combinations of genes are composed of a total of 12 combinations as seen in the following Table 4. A survey of diagnostic rates assessed by analyzing the expression patterns of the gene combinations in each cancer tissue was performed according to the 2003 WHO guidelines as described in Example 1. Also, the survey result is shown in the following Table 4.

TABLE 4

| | Correctly | Misclassi- | Indeterminate |
|---|---|---|---|
| | classified (%)^{a} | fied (%) | (%)^{b} |
| Two markers (n = 63) | | | |
| CK7/CK20 | 41 (65.1) | 3 (4.8) | 19 (30.2) |
| CK7/CDX2 | 45 (71.4) | 4 (6.3) | 14 (22.2) |
| CK7/MUC2 | 39 (61.9) | 5 (7.9) | 19 (30.2) |
| CK20/CDX2 | 41 (65.1) | 7 (11.1) | 15 (23.8) |
| CK20/MUC2 | 34 (54.0) | 7 (11.1) | 22 (34.9) |
| CDX2/MUC2 | 36 (57.1) | 6 (9.5) | 21 (33.3) |
| Three markers (n = 63) | | | |
| CK7/CK20/CDX2 | 51 (81.0) | 4 (6.3) | 8 (12.7) |
| CK7/CK20/MUC2 | 50 (79.4) | 3 (4.8) | 10 (15.9) |
| CK7/CDX2/MUC2 | 52 (82.5) | 4 (6.3) | 7 (11.1) |
| CK20/CDX2/MUC2 | 45 (71.4) | 3 (4.8) | 15 (23.8) |
| Four markers (n = 63) | | | |
| CK7/CK20/CDX2/MUC2 | 55 (87.3) | 4 (6.3) | 4 (6.3) |
| Seven markers (n = 63) | | | |
| CK7/CK20/CDX2/CEA/ | 47 (74.6) | 4 (6.3) | 12 (19.0) |
| MUC5AC/AMACR | | | |

³Cases with expression of more than two colonic markers classified as metastatic colorectal adenocarcinomas (MCAOs) and cases without any colonic marker classified as POMAs.
^{b}Cases with expression of one colonic marker considered unclassifiable.

The result of the survey of the diagnostic rates of MCAOs using one or more combinations of the genes selected in the present invention is shown in Table 4. Of the two-gene combinations, CK7/CDX2 showed the highest diagnostic rate of 71.4%. Among the three-gene combinations, CK20/CDX2/MUC2 gave a higher diagnostic rate (82.5%), compared to the other three-gene combinations. Among all the gene combinations, the four-gene combination of CK7/CK20/CDX2/MUC2 showed the highest diagnostic rate (87%). In contrast, the gene combination using all seven genes, i.e., CK7/CK20/CDX2/CEA/MUC2/MUC5AC/AMACR showed a lower diagnostic rate (74.6%) compared to the four-gene combinations.

Accordingly, from the above result, the present inventors have found that, when all of the four genes CK7/CK20/CDX2/MUC2 genes selected in the present invention are used, the onset of colorectal cancer can be effectively predicted and diagnosed, and further have found that the genes can be used as markers for distinguishing ovarian cancer from colorectal cancer.

Although the invention has been described focusing on the preferred embodiments, those skilled in the art will appreciate that the invention may be carried out in modified forms without departing from the essential characteristics of the present invention. Therefore, the above embodiments should be construed in all aspects as illustrative and not restrictive. The scope of the invention should be determined by the appended claims and their legal equivalents, not by the above description, and all changes coming within the equivalency range of the appended claims should be construed as being embraced in the invention.

What is claimed is:

1. A composition for diagnosing colorectal cancer or ovarian cancer, comprising an agent measuring mRNA or protein levels of two or more genes selected from the group consisting of CK7 (cytokeratin 7), CK20 (cytokeratin 20), CDX2 (caudal type homeobox transcription factor 2), and MUC2 (mucin 2).

2. The composition of claim 1, comprising an agent measuring mRNA or protein levels of a combination of genes selected from the group consisting of 1 to 8 listed in the following table:

| | gene combination |
|---|---|
| 1 | CK7/MUC2 |
| 2 | CK20/MUC2 |
| 3 | CDX2/MUC2 |
| 4 | CK7/CK20/MUC2 |
| 5 | CK7/CDX2/MUC2 |
| 6 | CK20/CDX2/MUC2 |
| 7 | CK7/CK20/CDX2/MUC2 |
| 8 | CK7/CK20/CDX2/CEA/MUC2/MUC5AC/AMACR |

3. The composition of claim 1, wherein the agent measuring mRNA levels of the genes is a primer or probe specifically binding to a gene selected from the group consisting of CK7, CK20, CDX2, and MUC2.

4. The composition of claim 1, wherein the agent measuring protein levels is an antibody specific to a protein selected from the group consisting of CK7, CK20, CDX2, and MUC2.

5. A kit for diagnosing colorectal cancer or ovarian cancer, comprising the composition of any of claim 1.

6. The kit of claim 5, wherein the kit is an RT-PCR kit, a DNA chip kit, or a protein chip kit.

7. A method for predicting and diagnosing colorectal cancer or ovarian cancer, comprising the steps of:
(a) measuring expression of two or more genes or proteins selected from the group consisting of CK7, CK20, CDX2, and MUC2 present in a biological sample; and
(b) comparing the measurement result in the step (a) with the expression of genes or proteins of normal control samples.

8. The method of claim 7, wherein the biological sample is selected from the group consisting of tissues, cells, whole blood, serum, plasma, saliva, and urine.

9. The method of claim 7, wherein the measurement is selected from the group consisting of reverse transcriptase-polymerase chain reaction, real time-polymerase chain reaction, Western blot, Northern blot, ELISA (enzyme linked immunosorbent assay), RIA (radioimmunoassay), radioimmunodiffusion, immunohistochemistry, and immunoprecipitation assay.

10. The method of claim 7, wherein, if the expression of the CK7 gene or protein is increased compared to the normal control samples and the expression of the CK20, CDX2, and MUC2 genes or proteins is decreased compared to the normal control samples, the onset of ovarian cancer is determined.

11. The method of claim 7, wherein, if the expression of the CK7 gene or protein is decreased compared to the normal control samples and the expression of the CK20, CDX2, and MUC2 genes or proteins is increased compared to the normal control samples, the onset of colorectal cancer is determined.
